(19) United States
(12) Plant Patent Application Publication
(10) Pub. No.: US 2015/0150181 P1
(43) Pub. Date: May 28, 2015

(54) Gaillardia plant named 'Sunset Popsy'

(50) ;

(71) Applicants: Keith George Lintott, Bognor Regis (GB); Charles Richard Read, Bognor Regis (GB)

(72) Inventors: Keith George Lintott, Bognor Regis (GB); Charles Richard Read, Bognor Regis (GB)

(21) Appl. No.: 14/544,086

(22) Filed: Nov. 21, 2014

Related U.S. Application Data
(60) Provisional application No. 61/963,168, filed on Nov. 25, 2013.

(51) Int. Cl. A01H 5/00

(52) U.S. Cl. PLT/431

(57) ABSTRACT

A new cultivar of Gaillardia named ‘Sunset Popsy’ that is distinguishable by a long blooming period, compact, low-growing habit and large inflorescences composed of two rows of flat ray florets which are dark-pink in color, with cream tips, is disclosed.

### CROSS REFERENCE TO RELATED APPLICATIONS

The present application claims priority to Provisional Application No. 61/963,168 filed on Nov. 25, 2013, entitled Gaillardia Plants Named ‘Celebration’, ‘Sunset Cutie’, ‘Sunset Flash’, ‘Sunset Mexican’, ‘Sunset Popsy’, ‘Sunset Sunrise’, ‘Sunset Snappy’, ‘Fanfare Regal’, ‘Fanfare Citronella’ and for which the inventor now wishes to file this application with the cultivar name or denomination ‘Sunset Popsy’ the entire contents of which is herein incorporated by reference in its entirety.

### GENUS AND SPECIES

Gaillardia×grandiflora

### VARIETY DENOMINATION

‘Sunset Popsy’

### BACKGROUND

The present invention relates to a new and distinct cultivar of blanket flower, an herbaceous perennial that is grown for use as an ornamental landscape and container plant. The new invention is known botanically as Gaillardia×grandiflora and will be referred to hereinafter by the cultivar name ‘Sunset Popsy’. Gaillardia is in the family Compositae, under which the commonly referred to “flower” is actually the inflorescence, and made up of smaller ray florets and disc florets. The ray florets themselves have the appearance of “petals”.

‘Sunset Popsy’ originated and was selected from a large population of hybrid seedlings from a Gaillardia breeding program which was started in 2006. The breeding program was conducted in a greenhouse nursery in West Sussex, United Kingdom. The aims of the breeding program are to produce novel combinations of flower colors and flower forms which are borne on well-branched plants with sturdy growth habits. A controlled hybridization was carried out using combinations of selected named varieties and selected unnamed and unreleased seedlings retained from previous breeding cycles.

‘Sunset Popsy’ was selected in 2009 for its dark-pink colored flat ray florets with cream tips which are produced from spring until fall. ‘Sunset Popsy’ was also selected for its compact and naturally branching habit.

The hybridization which led to the selection of ‘Sunset Popsy’ was carried out during 2008 using a proprietary Gaillardia unreleased seedling known as ‘G722-1’ (unpatented) as the male parent and a proprietary Gaillardia unreleased seedling known as ‘G722-3’ (unpatented) as the female parent.

‘Sunset Popsy’ was first vegetatively propagated in October 2009 at the inventors' nursery in West Sussex, United Kingdom using shoot cuttings taken from lateral branches. ‘Sunset Popsy’ has been found to reproduce true to type in successive generations of asexual propagation using shoot cuttings taken from lateral branches.

### SUMMARY

The following traits have been repeatedly observed and represent the distinguishing characteristics of ‘Sunset Popsy’. ‘Sunset Popsy’ has not been tested under all possible conditions and phenotypic differences may be observed with variations in environmental, climatic, and cultural conditions, without however, any variance in genotype.

1. ‘Sunset Popsy’ exhibits a compact and naturally branching plant habit.

2. ‘Sunset Popsy’ exhibits large single inflorescences composed of a two rows or layers of flat ray florets which are dark-pink in color with cream tips.

3. The disc of ‘Sunset Popsy’ is red in color, aging to deep red when the inflorescence is fully expanded.

4. ‘Sunset Popsy’ blooms continually from spring until late fall.

5. After one year's growth in a 3 liter container, ‘Sunset Popsy’ is 35 cm to 40 cm in height and 40 cm to 45 cm in width.

6. ‘Sunset Popsy’ is hardy in USDA zone 5.

### DESCRIPTION OF THE PHOTOGRAPHS

The accompanying color photographs illustrate the overall appearance of the new Gaillardia cultivar ‘Sunset Popsy’ showing the colors as true as it is reasonably possible to obtain in colored reproductions of this type. Colors in the photographs may differ from the color values cited in the detailed botanical description, which more accurately describes the actual colors of the new variety ‘Sunset Popsy’.

FIG. 1 depicts a whole plant in bud of ‘Sunset Popsy’ which has been grown entirely out of doors in Oxnard, Calif. The illustrated plant is seven-months old and has been grown in a 30 cm diameter container without any pinching or chemical growth regulator.

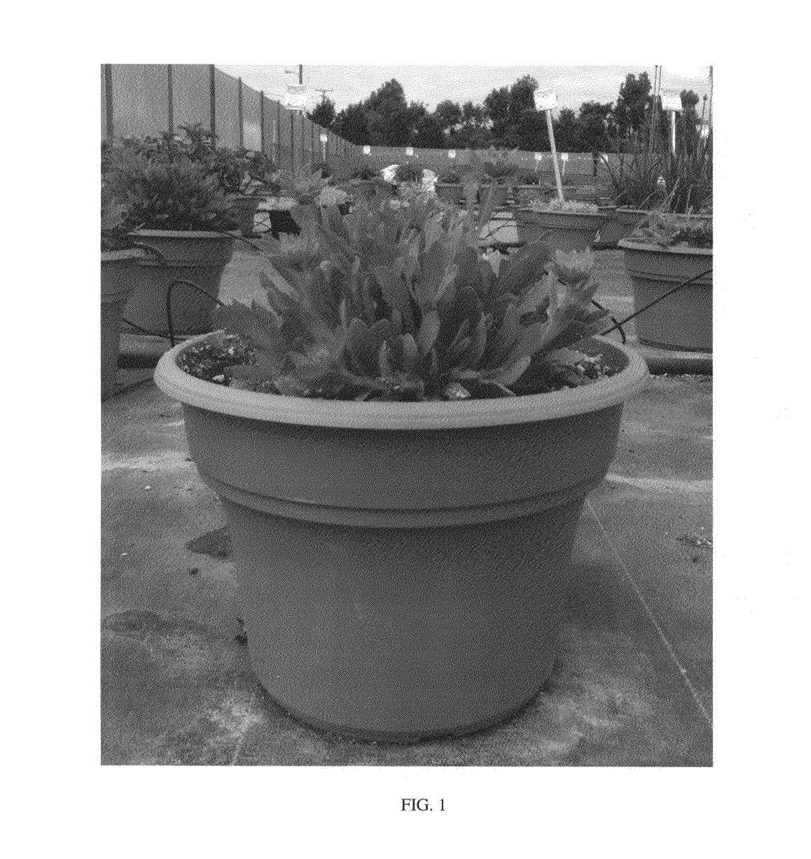

FIG. 2 depicts a close up view of the fully expanded inflorescences of ‘Sunset Popsy’.

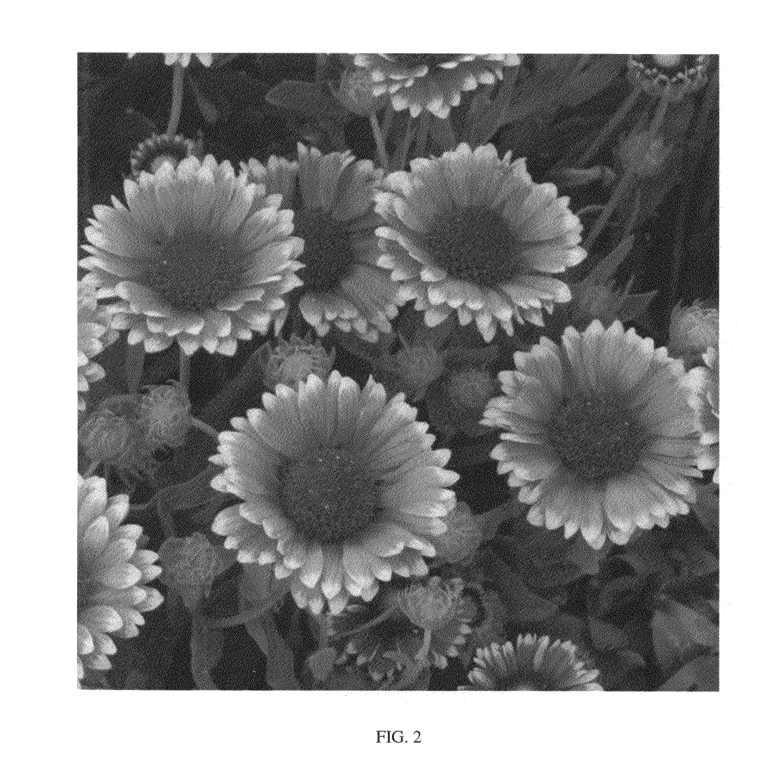

### DESCRIPTION OF THE NEW VARIETY

The following detailed descriptions set forth the distinctive characteristics of ‘Sunset Popsy’. Observations, measurements, values and comparisons were compiled during October 2014 in Santa Barbara, Calif. from a one-year old plant growing out of doors in full sun in freely drained proprietary peat-based potting soil. Color determinations are made in accordance with the 2007 edition of The Royal Horticultural Society Colour Chart from London England, except where general color terms of ordinary dictionary significance are used.
Classification:
   Family.—Compositae.
   Genus.—Gaillardia.
   Species.—×grandiflora.
   Common name.—Blanket flower.
Parentage:
   Male parent.—Unreleased proprietary Gaillardia seedling, code number ‘G722-1’ (unpatented).
   Female parent.—Unreleased proprietary Gaillardia seedling, code number ‘G722-3’ (unpatented).
Plant:
   Propagation method.—Typically by softwood cuttings although root cuttings have also been successful.
   Rooting system.—Fine and fibrous.
   Vigor.—Moderate vigor.
   Time to develop roots.—14 to 20 days are needed for an initial cutting to develop roots.
   Temperature to develop roots.—The recommended air temperature is 20-21° Centigrade.
   Crop time.—Approximately 10 weeks to 2 months are needed to produce a 15-centimeter container from a rooted cutting.
   Growth habit.—Compact and naturally freely branching.
   Suggested container size.—15-centimeter container.
   Use.—Ornamental for use as a landscape plant or container plant.
   Type.—Herbaceous perennial.
   Overall dimensions.—After one year's growth in a 3 liter container: 35 cm to 40 cm in height and 40 cm to 45 cm in width.
   At maturity in the ground.—40 cm to 45 cm in height and 50 cm to 55 cm in width.
   Cultural requirements.—Grow in full sun with moderate water, and well-draining soil such as loam.
   Hardiness.—USDA Zone 5.
   Growing requirements.—If grown outside in regions which experience winter freezing, ‘Sunset Popsy’ may be started as a cutting during the spring or summer of the previous year, planted out prior to the onset of winter, and flowering will commence typically in May or June according to the region and season. When grown outside in frost-free regions, or in frost-protected greenhouses, ‘Sunset Popsy’ will flower virtually all year round and may be started as a cutting (which should be non-flowering) at any time of year.
Stems:
   Branching habit.—Basal branching.
   Stem color.—138B.
   Stem dimensions.—10 cm to 15 cm in length and 5 mm in width.
   Stem shape.—Cylindrical, slightly fluted longitudinally.
   Stem surface.—Markedly villous with dense coverage of very fine silvery hairs, approximately 3 mm to 4 mm in length.
   Internode length.—Average internode length is 10 mm to 12 mm.
Foliage:
   Type.—Evergreen.
   Leaf arrangement.—Alternate.
   Margin.—Entire.
   Leaf shape.—Oblanceolate.
   Leaf dimensions (base of plant).—20 cm to 23 cm in length, 3.5 cm in width.
   Leaf dimensions (at inflorescence height).—8 cm to 9 cm in length, 2 cm in width.
   Leaf base.—Truncate.
   Leaf apex.—Rounded.
   Leaf attachment.—Sessile.
   Leaf color (both surfaces).—138A.
   Leaf venation pattern.—Parallel; mid vein protrudes on both surfaces.
   Vein color (both surfaces).—138B.
   Leaf surface (both surfaces).—Pubescent, very fine silvery hairs, lighter than 156D, approximately 3 mm in length.
   Fragrance.—A slight sage-like scent when bruised.
Inflorescence:
   Inflorescence.—Solitary.
   Aspect.—Facing upward.
   Dimensions of inflorescence.—80 mm to 85 mm in diameter and 15 mm in height.
   Inflorescence type.—Radiate capitate with central disc.
   Disc diameter (inflorescence fully expanded).—25 mm to 30 mm.
   Inflorescence number per plant.—A one-year old plant bears approximately 25 to 35 inflorescences in bud and flower at one time.
   Blooming season.—Spring, summer and fall.
   Lastingness of inflorescence on the plant.—10 days, reducing to 7 days in full sun in mid-summer.
Peduncle:
   Peduncle dimensions.—8 cm to 11 cm in length and 2.5 mm in diameter.
   Peduncle shape.—Cylindrical.
   Peduncle surface.—Surface is pubescent and exhibits longitudinal ridges.
   Peduncle color.—138C.
   Peduncle strength.—Stiff.
Flower buds:
   Bud shape.—Rotate whorl.
   Bud dimensions (bud).—13 mm to 15 mm in diameter and 8 cm in height.
   Bud color.—138C.
   Bud surface.—Villous.
Ray florets:
   Ray floret shape.—Fan shaped with 3 fused petals appearing as lobes.
   Ray floret surface (adaxial surface).—Glabrous.
   Ray floret surface (abaxial surface).—Pubescent.
   Ray floret color (adaxial surface).—51A extending from petal base to within 4 mm to 5 mm of petal apex, where color changes to 16B extending to petal apex.
   Ray floret color (abaxial surface).—180D extending from petal base to within 4 mm to 5 mm of petal apex, where color changes to 16D extending to petal apex.
   Ray floret arrangement.—Radiate.
   Number of ray florets per inflorescence.—15 to 18 ray florets per inflorescence.
   Number of petals per ray floret.—Three.
   Fused or unfused.—Petals are basally fused.
   Petal margins of ray florets.—Entire.
   Ray floret dimensions.—30 mm to 35 mm in length including the corolla tube, 18 mm to 22 mm in width at the widest point.
   Corolla tube dimensions.—3 mm to 5 mm in depth and 0.8 mm to 1.0 mm in diameter.
   Ray floret veins (both surfaces).—Approximately three longitudinal, parallel, fine but prominent veins, color 53B.
   Self-cleaning or persistent.—Self-cleaning.
Disc florets:
   Quantity of disc florets per inflorescence.—Numerous, approximately 200 to 250.
   Disc floret dimensions (including pistil length).—9 mm in length and 2.5 mm in width.
   Depth of disc floret corolla tube.—6 mm in depth.
   Surface of disc florets.—Lanate.
   Color of disc florets (inflorescence newly opening, both surfaces).—45B.
   Color of disc florets (inflorescence fully expanded, both surfaces).—Ranges between 46A and 53A.
   Phyllary dimensions.—5.50 cm in diameter and 3 cm in length.
   Phyllary color.—147B.
   Phyllary arrangement.—Whorl.
   Number of involucral bracts.—An average of 25 in number per inflorescence.
   Shape of involucral bract.—Oblanceolate.
   Involucral bract dimensions.—2.0 cm in length and 0.5 cm to 0.8 cm in width.
   Involucral bract margin.—Entire.
   Involucral bract apex.—Acute.
   Involucral bract base.—Truncate.
   Involucral bract color (both surfaces).—138B.
   Involucral bract surface (abaxial surface).—Pubescent.
   Involucral bract surface (adaxial surface).—Pubescent.
   Fragrance of inflorescence.—Sweet fragrance.
Reproductive organs:
   Stamens (present on disc florets only).—Three in number, adnate to inner surface of corolla tube.
   Stamen dimensions.—0.20 mm in width and 5 mm in length.
   Stamen color.—17A.
   Stamen form.—Plumose.
   Anther dimensions.—0.50 mm in length and 1 mm in width.
   Anther color.—187A.
   Anther shape.—Narrow lanceolate.
   Pollen.—Present.
   Quantity of pollen.—Large amount.
   Pollen color.—17B.
   Pistil (present on ray florets and disc florets).—One per floret.
   Pistil length.—12 mm in length.
   Style dimensions.—2 mm in length and 1 mm in width.
   Style color.—150D.
   Stigma dimensions.—8 mm in length and 1.75 mm in width.
   Stigma form.—Plumose.
   Stigma color.—187A.
   Stigma shape.—Bifurcate.
   Ovary position.—Inferior.
   Ovary color.—150D.
   Ovary shape.—Globose.
   Ovary dimensions.—1 mm in width and 2 mm in height.
Fruit/seed set:
   Number of seeds.—Small to moderate amount (ranges from 50 to 100 seeds per inflorescence).
   Seed dimensions.—5 mm to 6 mm in length (including hairs at apex) and 1 mm to 2 mm in diameter.
   Seed color.—200A.
   Seed shape.—Conical with rounded base and apex.
   Seed surface.—Smooth except at apex where many very fine silver hairs, up to 2 mm in length, are present in circular groups.
Disease and pest susceptibility: ‘Sunset Popsy’ has not been observed to exhibit any resistance to any particular pest or disease. ‘Sunset Popsy’ is susceptible to downy mildew and to thrips as may be typical of Gaillardia.

### COMPARISON WITH PARENTAL LINES AND KNOWN VARIETIES

‘Sunset Popsy’ is distinguishable from the female parent, ‘G722-3’ as follows. Whereas the female parent exhibits carmine-pink flat ray florets with yellow tips, the ray florets of ‘Sunset Popsy’ are dark-pink in color with cream tips. Additionally, ‘Sunset Popsy’ has a more open growth habit and bears more flowers than that of the female parent.

‘Sunset Popsy’ is distinguishable from the male parent, ‘G722-1’ as follows. Whereas the flowers of ‘Sunset Popsy’ consist of two rows (layers) of flat ray florets, the ray florets of the male parent are arranged in one row (layer) of flat petals. Additionally ‘Sunset Popsy’ exhibits a longer flowering period and a shorter growth habit than the male parent.

The variety of Gaillardia which is considered to most closely resemble ‘Sunset Popsy’ is the variety Gaillardia ‘Burgunder’ (unpatented) which is also a hardy variety of Gaillardia. Whereas the ray florets of ‘Sunset Popsy’ are predominantly dark-pink in color with cream tips and arranged in two rows, the ray florets of ‘Burgunder’ are wine-red in color and are arranged in a single row. Additionally, ‘Sunset Popsy’ has a shorter and more compact habit and flowers over a longer period than ‘Burgunder’.

‘Sunset Popsy’ may also be compared with the variety, Gaillardia ‘Oranges and Lemons’ (U.S. Plant Pat. No. 17,092) which is also a hardy variety of Gaillardia. The flowers of ‘Oranges and Lemons’ are orange and yellow in color, whereas the flowers of ‘Sunset Popsy’ are dark pink in color with cream tips. Additionally, the flowers of ‘Sunset Popsy’ are more abundant than those of ‘Oranges and Lemons’. Further, the plant habit of ‘Sunset Popsy’ is more compact than that of ‘Oranges and Lemons’.

We claim:

1. A new and distinct cultivar of Gaillardia plant named ‘Sunset Popsy’ as described and illustrated herein.
